(19) United States
(12) Patent Application Publication
(10) Pub. No.: US 2026/0096847 A1
(43) Pub. Date: Apr. 9, 2026

(54) ABLATION DEVICE AND ABLATION SYSTEM

(71) Applicant: INDUSTRIAL TECHNOLOGY RESEARCH INSTITUTE, Hsinchu (TW)

(72) Inventors: Jo-Ping LEE, Hsinchu City (TW); Chung-Hsin SU, Hsinchu City (TW)

(73) Assignee: INDUSTRIAL TECHNOLOGY RESEARCH INSTITUTE, Hsinchu (TW)

(21) Appl. No.: 19/005,127

(22) Filed: Dec. 30, 2024

(30) Foreign Application Priority Data
Oct. 9, 2024 (TW) 113138386

(51) Int. Cl. A61B 18/14; A61B 18/00

(52) U.S. Cl. CPC A61B 18/1477; A61B 2018/00541; A61B 2018/00577; A61B 2018/00773; A61B 2018/00791

(57) ABSTRACT

An ablation device and system, where the ablation device includes a penetrating needle and an angle adjusting assembly. The penetrating needle is movably disposed through the sheath. A part of the penetrating needle is flexible. The angle adjusting assembly includes a clamping component disposed between the sheath and the penetrating needle. The clamping component has a clamping state and a releasing state. When the clamping component is in the clamping state, the clamping component is configured to support the penetrating needle to allow the penetrating needle to be configured to penetrate into the affected part along an axial direction of the sheath, and the clamping component is configured to adjust a penetrating angle of the penetrating needle. When the clamping component is in the releasing state, the clamping component releases the penetrating needle.

### CROSS-REFERENCE TO RELATED APPLICATIONS

All related applications are incorporated by reference. The present application is based on, and claims priority from, Taiwan (International) Application Serial Number 113138386 filed on Oct. 9, 2024, the disclosure of which is hereby incorporated by reference herein in its entirety.

### TECHNICAL FIELD

The disclosure relates to an ablation device and an ablation system.

### BACKGROUND

In recent years, Radio frequency ablation (RFA) is the most widely used tumor ablation technique. During the RFA, with the assistance of the computed tomography (CT) images, a doctor can move a rigid needle of an ablation device to penetrate into a lung with tumor through a skin.

### SUMMARY

One embodiment of this disclosure provides an ablation device configured to be cooperated with a sheath and penetrate into an affected part. The ablation device includes a penetrating needle and an angle adjusting assembly. The penetrating needle is movably disposed through the sheath. A part of the penetrating needle is flexible. The angle adjusting assembly includes a clamping component disposed between the sheath and the penetrating needle. The clamping component has a clamping state and a releasing state. When the clamping component is in the clamping state, the clamping component is configured to support the penetrating needle to allow the penetrating needle to be configured to penetrate into the affected part along an axial direction of the sheath, and the clamping component is configured to adjust a penetrating angle of the penetrating needle. When the clamping component is in the releasing state, the clamping component releases the penetrating needle.

Another embodiment of this disclosure provides an ablation system configured to be cooperated with a sheath and penetrate into an affected part. The ablation system includes a control module, an ablation apparatus and an ablation device. The ablation apparatus is electrically connected to the control module. The ablation device includes a penetrating needle and an angle adjusting assembly. The penetrating needle is movably disposed through the sheath. A part of the penetrating needle is flexible. The penetrating needle is electrically connected to the ablation apparatus. The control module is configured to control the ablation apparatus to send an ablation signal to the penetrating needle. The angle adjusting assembly includes a clamping component disposed between the sheath and the penetrating needle. The clamping component has a clamping state and a releasing state. When the clamping component is in the clamping state, the clamping component is configured to support the penetrating needle to allow the penetrating needle to be configured to penetrate into the affected part along an axial direction of the sheath, and the clamping component is configured to adjust a penetrating angle of the penetrating needle. When the clamping component is in the releasing state, the clamping component releases the penetrating needle.

### BRIEF DESCRIPTION OF THE DRAWINGS

The present disclosure will become better understood from the detailed description given herein below and the accompanying drawings which are given by way of illustration only and thus are not intending to limit the present disclosure and wherein:

FIG. 1 is a block diagram of an ablation system according to a first embodiment of the disclosure;

FIG. 2 is a cross-sectional view of an ablation device in FIG. 1;

FIG. 3 is a cross-sectional view of the ablation device in FIG. 2 taken along line 3-3;

FIG. 4 is a cross-sectional view of the ablation device in FIG. 2 taken along line 4-4;

FIG. 5 is a cross-sectional view of the ablation device in FIG. 2 taken along line 5-5;

FIG. 6 is a cross-sectional view showing that a penetrating needle of the ablation device in FIG. 2 is moved into a sheath;

FIG. 7 is a cross-sectional view showing that a clamping component of the ablation device in FIG. 2 is in a clamping state;

FIG. 8 is the ablation device in FIG. 7 from another viewing angle;

FIGS. 9 and 10 are cross-sectional views showing that the penetrating needle of the ablation device in FIG. 7 penetrates into an affected part;

FIG. 11 is a cross-sectional view showing that a penetrating angle of the penetrating needle of the ablation device in FIG. 1 is adjusted;

FIG. 12 is a cross-sectional view of the ablation device in FIG. 11 from another viewing angle;

FIG. 13 is a cross-sectional view of an ablation device according to a second embodiment of the disclosure;

FIG. 14 is a partially enlarged perspective view of the ablation device in FIG. 13;

FIG. 15 is a plan view of the ablation device in FIG. 13 from another viewing angle;

FIG. 16 is a cross-sectional view of an ablation device according to a third embodiment of the disclosure;

FIG. 17 is a partially enlarged perspective view of the ablation device in FIG. 16;

FIG. 18 is a plan view of the ablation device in FIG. 16 from another viewing angle;

FIG. 19 is a cross-sectional view of an ablation device according to a fourth embodiment of the disclosure;

FIG. 20 is a partially enlarged perspective view of the ablation device in FIG. 19; and

FIG. 21 is a plan view of the ablation device in FIG. 19 from another viewing angle.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

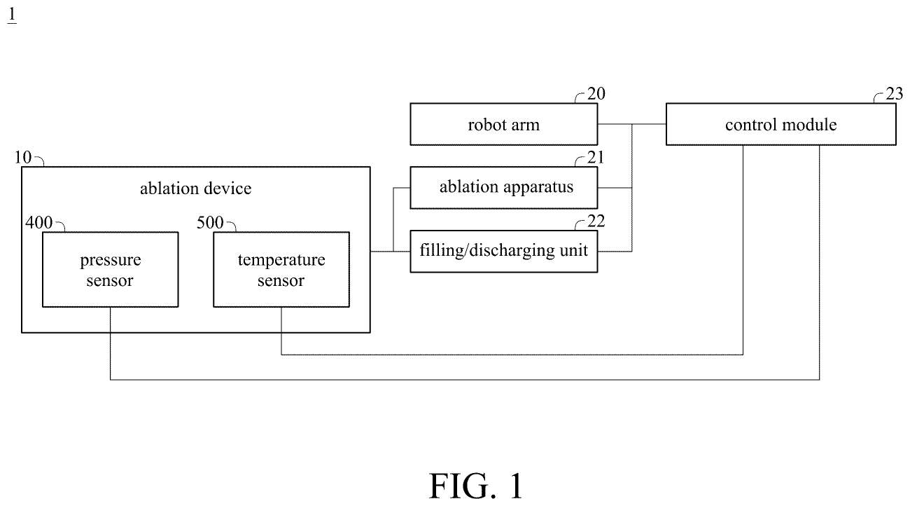

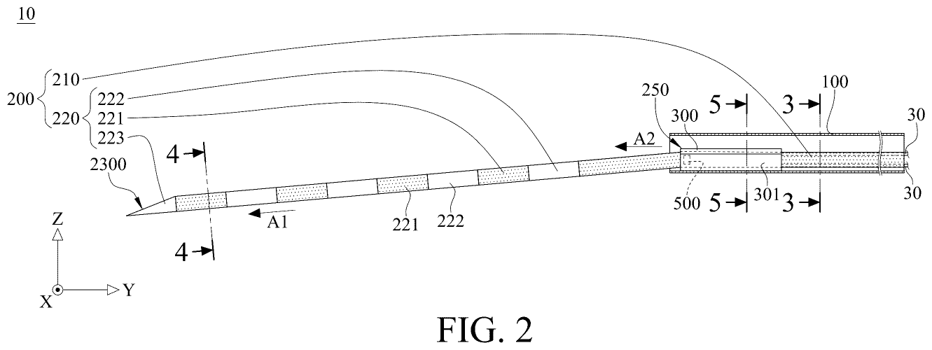

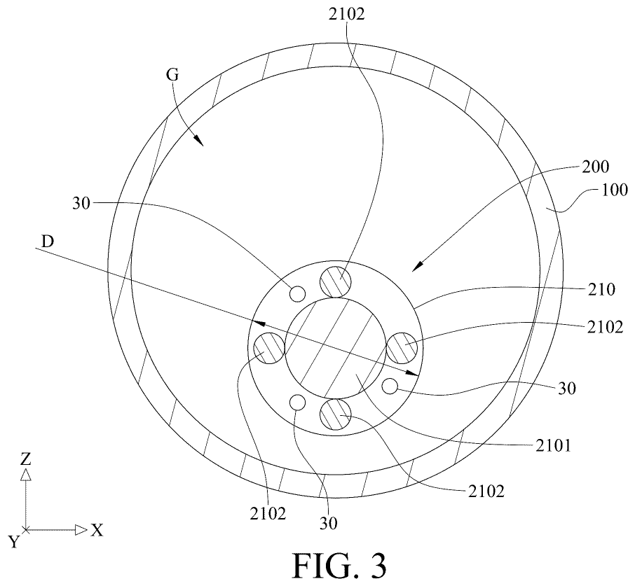

Please refer to FIGS. 1 to 3. FIG. 1 is a block diagram of an ablation system 1 according to a first embodiment of the disclosure. FIG. 2 is a cross-sectional view of an ablation device 10 in FIG. 1. FIG. 3 is a cross-sectional view of the ablation device 10 in FIG. 2 taken along line 3-3.

In this embodiment, the ablation system 1 includes the ablation device 10, a robot arm 20, an ablation apparatus 21, a filling/discharging unit 22 and a control module 23. The control module 23 is electrically connected to the robot arm 20, the ablation apparatus 21 and the filling/discharging unit 22. The filling/discharging unit 22 is, for example, a pump.

In this embodiment, the ablation device 10 includes a penetrating needle 200, an angle adjusting assembly 250, a plurality of pressure sensors 400 and a temperature sensor 500, and is configured to be cooperated with a sheath 100.

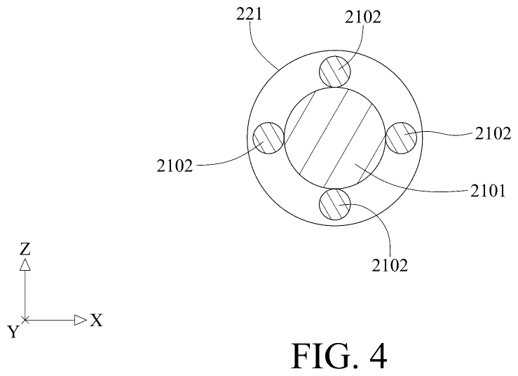

Please refer to FIGS. 2 to 4. FIG. 3 is a cross-sectional view of the ablation device 10 in FIG. 2 taken along line 3-3. FIG. 4 is a cross-sectional view of the ablation device 10 in FIG. 2 taken along line 4-4.

The penetrating needle 200 is movably disposed through the sheath 100, and a part of the penetrating needle 200 is flexible. In an embodiment, the sheath 100 may be a sheath of an endoscope. In detail, in this embodiment, the penetrating needle 200 has an assembling end part 210 and a penetrating end part 220 that are connected to each other. The assembling end part 210 is movably disposed in the sheath 100, and is, for example, flexible.

Further, as shown in FIG. 3, in this embodiment, for example, a supporting post 2101, a plurality of wires 2102 and a plurality of tubes 30 are disposed in the assembling end part 210. The wires 2102 and the tubes 30, for example, surround the supporting post 2101. An insulating material may be filled between the assembling end part 210 and the supporting post 2101 to support the wires 2102 and the tubes 30.

In addition, as shown in FIG. 3, in this embodiment, a maximum diameter D of the penetrating needle 200 is, for example, smaller than or equal to 1.5 millimeter (mm).

The penetrating end part 220 includes a plurality of flexible portions 221, a plurality of rigid portions 222 and a penetrating portion 223. The flexible portions 221 and the rigid portions 222 are alternately arranged and connected along an axial direction A1 of the penetrating end part 220. The penetrating portion 223 protrudes from the flexible portion 221 that is located farthest away from the assembling end part 210, and has a penetrating inclined surface 2300 located on a free end thereof. In addition, in this embodiment, for example, the flexible portion 221 is electrically insulating and the rigid portion 222 is electrically conductive. Further, the rigid portions 222 are, for example, electrodes, and the rigid portions 222 are configured to together define an ablation region. Since the penetrating end part 220 of the penetrating needle 200 has multiple portions (i.e., the flexible portions 221 and the rigid portions 222) that are arranged alternately, the ablation device 10 is allowed to enter into a bronchus to perform an ablation surgery by cooperating with an endoscope. In this way, the ablation surgery is allowed to be performed without the assistance of the computed tomography (CT) images, which prevents the patient from being exposed to the radiation. Moreover, the possibility of bringing the medical complication (e.g., pneumothorax) after performing the ablation by the ablation device 10 is lowered.

The disclosure is not limited by the number of the flexible portions 221 and the number of the rigid portions 222. In other embodiments, the penetrating end part may include one flexible portion and one rigid portion.

Furthermore, in this embodiment, as shown in FIG. 4, the supporting post 2101 and the wires 2102 are also disposed in, for example, the flexible portions 221 and the rigid portions 222. For the brevity, it is shown in the drawings that the supporting post 2101 and the wires 2102 are disposed in the flexible portions 221, and it is omitted from the drawings that the supporting post 2101 and the wires 2102 are disposed in the rigid portions 222. An insulating material may be filled, for example, between the flexible portions 221 and the supporting post 2101 to support the wires 2102. In addition, the wires 2102 are electrically connected to, for example, the rigid portions 222 and the ablation apparatus 21 in FIG. 1.

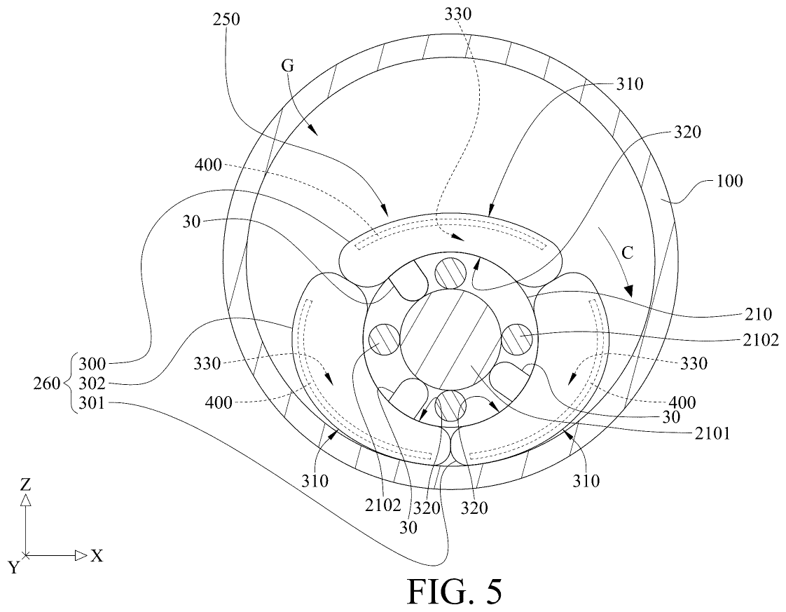

Please refer to FIGS. 2 and 5. FIG. 5 is a cross-sectional view of the ablation device 10 in FIG. 2 taken along line 5-5. In this embodiment, the angle adjusting assembly 250 includes a clamping component 260. The clamping component 260 includes, for example, a plurality of elastic bags 300, 301 and 302. The elastic bags 300, 301 and 302 are disposed between the sheath 100 and the assembling end part 210 of the penetrating needle 200. The elastic bags 300, 301 and 302 each have a fixed surface 310, a movable surface 320 and a chamber 330, where the fixed surface 310 and the movable surface 320 face away from each other, and the chamber 330 is an internal chamber. The fixed surface 310 faces the sheath 100. The movable surface 320 faces the assembling end part 210. The fixed surface 310 and the movable surface 320 face away from the chamber 330. The elastic bags 300, 301 and 302 are sleeved on and together surround the assembling end part 210. In addition, the elastic bags 300, 301 and 302 are arranged along a circumferential direction C of the assembling end part 210. The chambers 330 of the elastic bags 300, 301 and 302 are not in fluid communication with one another. Moreover, the elastic bags 300, 301 and 302 are in fluid communication with the filling/discharging unit 22 via, for example, three tubes 30, respectively. Also, the surfaces of the elastic bags 300, 301 and 302 are made of, for example, electrically insulating material.

It should be understood that although the tubes 30 and the filling/discharging unit 22 are external to the ablation device 10 in this embodiment, in other embodiments, the clamping component, the tubes and the filling/discharging unit may together configure the angle adjusting assembly of the ablation device.

The clamping component 260 in FIGS. 2 and 5 is in a releasing state where the elastic bags 300, 301 and 302 are not filled with fluid and thus do not sufficiently support the penetrating needle 200 (i.e., the elastic bags 300, 301 and 302 are released).

In this embodiment, for example, since the elastic bags 300, 301 and 302 are allowed to be filled or released to be deformed correspondingly, the clamping component 260 including the elastic bags 300, 301 and 302 has, for example, elasticity. However, in other embodiments, the clamping component may be a structure without elasticity, as long as the clamping component can sufficiently support the penetrating needle in a clamping state.

In other embodiments, the clamping component may include one elastic bag sleeved on the penetrating needle. That is, the disclosure is not limited by the number of the elastic bag(s). The number of the elastic bag(s) may be adjusted to be one or more as long as the elastic bag(s) is/are disposed between the penetrating needle and the sheath to sufficiently support the penetrating needle in the clamping state. Moreover, in other embodiments, the elastic bag(s) may be fixed to the sheath instead of being sleeved on the penetrating needle.

Please refer to FIGS. 1, 2 and 5. The pressure sensors 400 are electrically connected to the control module 23. The pressure sensors 400 are in, for example, a thin film configuration, and disposed on sides of the elastic bags 300, 301 and 302 located close to the sheath 100, respectively. For example, the pressure sensors 400 sense the pressure in the chambers 330 of the elastic bags 300, 301 and 302, respectively. In this way, the control module 23 is allowed to determine the filling states of the elastic bags 300, 301 and 302. Thus, the functional operation of the elastic bags 300, 301 and 302 is allowed to be ensured or the penetrating angle is allowed to be adjusted according to the filling states.

The temperature sensor 500 is electrically connected to the control module 23, and is disposed on a side of each of the elastic bags 300, 301 and 302. For example, the temperature sensor 500 senses the temperature of the ablation device 10 and senses the ablation range, thereby preventing the endoscope cooperating with the ablation device 10 from being damaged due to high temperature. Specifically, the control module 23 receives the temperature data sensed by the temperature sensor 500, and control the power of the ablation signal sent from the ablation apparatus 21 based on the temperature data. For example, if the temperature sensor 500 senses that the temperature of the ablation device 10 is high, the control module 23 will decrease the power of the ablation signal sent from the ablation apparatus 21; if the temperature sensor 500 senses that the temperature of the ablation device 10 is low, the control module 23 will increase the power of the ablation signal sent from the ablation apparatus 21.

In other embodiments, if there is no demand for sensing the pressure and/or the temperature, the ablation device may not include the pressure sensor and/or the temperature sensor.

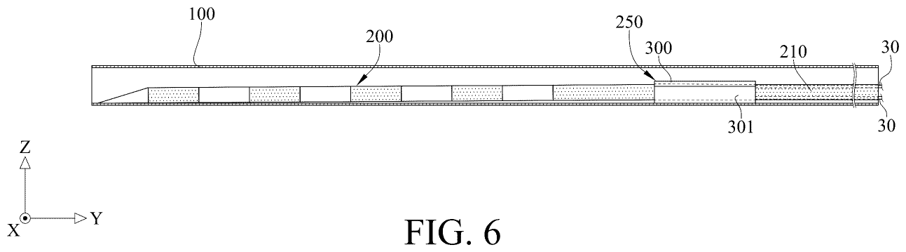

Please refer to FIGS. 1, 2 and 6. FIG. 6 is a cross-sectional view showing that the penetrating needle 200 of the ablation device 10 in FIG. 2 is moved into the sheath 100. Before the ablation device 10 is moved to an ablation position as shown in FIG. 2, the ablation device 10 is required to be inserted into, for example, the bronchus. When the ablation device 10 is moved to the ablation position in the bronchus, the penetrating needle 200 is moved into the sheath 100 as shown in FIG. 6. In this way, the penetrating needle 200 is prevented from being in unnecessary contact with the bronchus or other adjacent organs. When the ablation device 10 is moved to the ablation position, the control module 23 controls the robot arm 20 to push the penetrating needle 200 out of the sheath 100 along an axial direction A2 of the sheath 100, thereby facilitating the following ablation process. Note that the clamping component 260 in FIG. 6 may be in the releasing state.

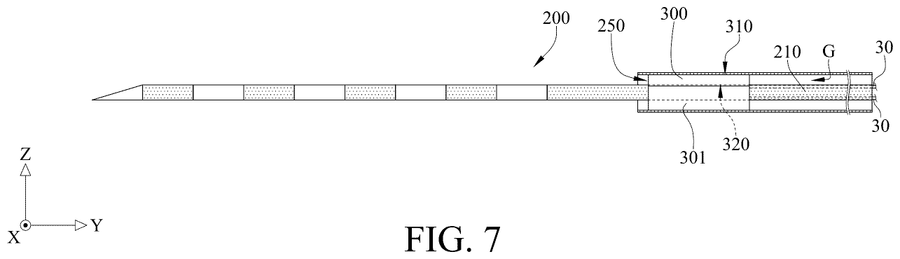

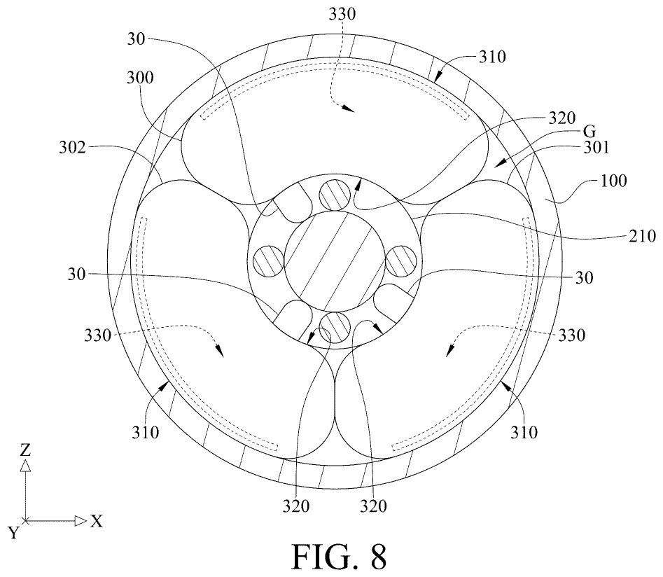

Please refer to FIGS. 1, 7 and 8. FIG. 7 is a cross-sectional view showing that the clamping component 260 of the ablation device 10 in FIG. 2 is in a clamping state. FIG. 8 is the ablation device 10 in FIG. 7 from another viewing angle. The control module 23 controls the filling/discharging unit 22 to fill the elastic bags 300, 301 and 302 via the tubes 30 to expand the same, thereby allowing the clamping component 260 to be in the clamping state. For example, the filling/discharging unit 22 may fill a fluid, such as a gas or a liquid, into the chambers 330 of the elastic bags 300, 301 and 302 via the tubes 30 to expand the same. When the elastic bags 300, 301 and 302 expand (i.e., the chambers 330 are filled with fluid) and fills a gap G between the sheath 100 and the assembling end part 210, the fixed surface 310 and the movable surface 320 rest on the sheath 100 and the assembling end part 210, respectively. In this way, the elastic bags 300, 301 and 302 sufficiently supports the assembling end part 210, thereby changing the penetrating needle 200 from a sag state shown in FIG. 2 to a horizontal state shown in FIG. 7.

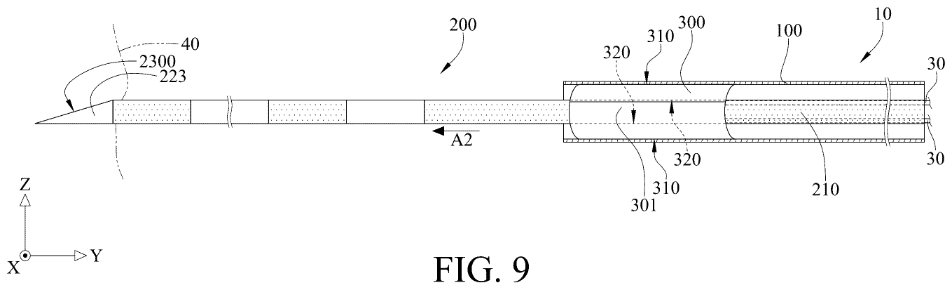

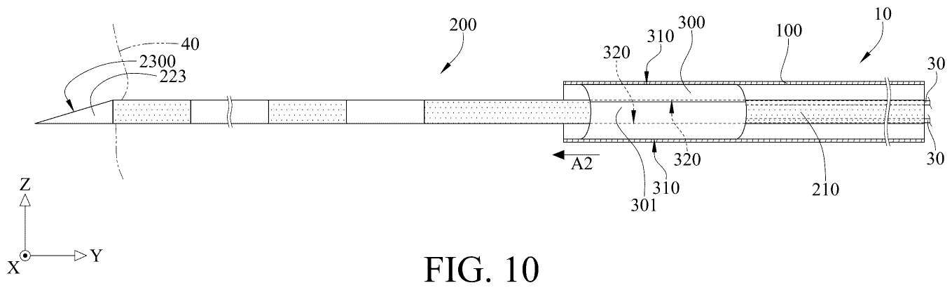

Please refer to FIGS. 1, 9 and 10. FIGS. 9 and 10 are cross-sectional views showing that the penetrating needle 200 of the ablation device 10 in FIG. 7 penetrates into an affected part 40. The control module 23 of this disclosure controls the robot arm 20 to force the penetrating needle 200 to penetrate into the affected part 40 in two manners. Firstly, the first manner is described. Please refer to FIG. 9. When the control module 23 controls the robot arm 20 to move the penetrating needle 200 along the axial direction A2 of the sheath 100, the penetrating portion 223 of the penetrating needle 200 penetrates into the affected part 40 via the penetrating inclined surface 2300, and the movable surfaces 320 of the elastic bags 300, 301 and 302 are moved along the axial direction A2 relative to the fixed surfaces 310 together with the penetrating needle 200, thereby allowing the elastic bags 300, 301 and 302 to be deformed.

Then, the second manner is described. Please refer to FIG. 10. When the control module 23 controls the robot arm 20 to move the sheath 100 along the axial direction A2, the fixed surfaces 310 of the elastic bags 300, 301 and 302 are moved along the axial direction A2 relative to the movable surfaces 320 together with the sheath 100, thereby allowing the elastic bags 300, 301 and 302 to be deformed. In this way, the sheath 100 moves the penetrating needle 200 along the axial direction A2 via the elastic bags 300, 301 and 302, thereby allowing the penetrating portion 223 of the penetrating needle 200 to penetrate into the affected part 40 of the penetrating inclined surface 2300. Note that regardless of the penetrating angle between the penetrating needle 200 and the sheath 100, the penetrating needle 200 should be understood as penetrating into the affected part 40 along the axial direction A2 as long as the penetrating needle 200 is displaced along the axial direction A2 to penetrate into the affected part 40.

Note that even though a part of the penetrating needle 200 is flexible, the elastic bags 300, 301 and 302 still provide sufficient support during, for example, expansion, thereby allowing the penetrating needle 200 to penetrate into the affected part 40 to perform ablation surgery.

In addition, when the penetrating portion 223 penetrates into the affected part 40 via the penetrating inclined surface 2300, the control module 23 may control the ablation apparatus 21 to send the ablation signal to the ablation device 10, and thus the ablation signal is sent to the penetrating portion 223 of the penetrating needle 200 to enable the ablation surgery to be performed on the affected part 40.

After the ablation surgery is completed, the control module 23 may control the robot arm 20 to move the penetrating needle 200 back along a direction opposite to the axial direction A2, and may control the filling/discharging unit 22 to release the elastic bags 300, 301 and 302 via the tubes 30 (e.g., discharging the fluid out of each of the elastic bags 300, 301 and 302 to allow a part of the fixed surface 310 to be spaced apart from the sheath 100), thereby changing the ablation device 10 back into the state shown in FIG. 2.

Note that the disclosure is not limited to controlling the ablation device 10 by the robot arm 20. In other embodiments, the ablation system may not include the robot arm, and the penetrating needle or the sheath may be controlled manually.

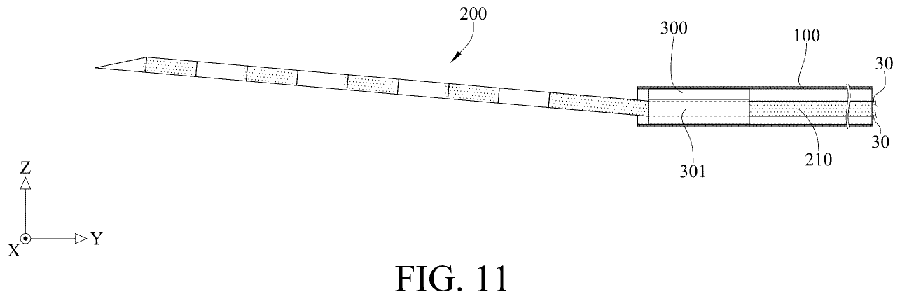

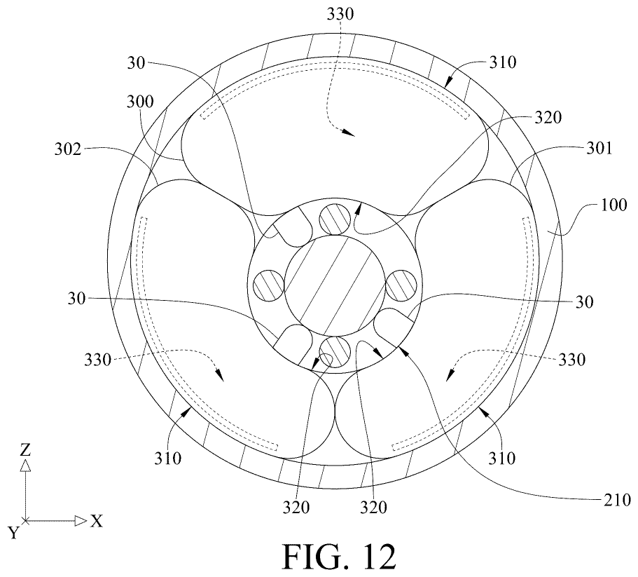

Please refer to FIGS. 1, 11 and 12. FIG. 11 is a cross-sectional view showing that a penetrating angle of the penetrating needle 200 of the ablation device 10 in FIG. 1 is adjusted. FIG. 12 is a cross-sectional view of the ablation device 10 in FIG. 11 from another viewing angle. When the penetrating angle of the penetrating needle 200 to the sheath 100 is required to be adjusted, the control module 23 may control the filling/discharging unit 22 to differently fill and/or release the elastic bags 300, 301 and 302 via the tubes 30 based on the pressure data sensed by the pressure sensors 400. For example, the filling/discharging unit 22 may allow the elastic bag 300 at a higher position (i.e., located above the elastic bags 301 and 302 along Z-axis direction) to expand more than the elastic bags 301 and 302. Accordingly, an end of the penetrating needle 200 is lifted up along positive Z-axis direction. That is, in this disclosure, based on the pressure data sensed by the pressure sensors 400, the elastic bags 300, 301 and 302 may expand differently, thereby adjusting the penetrating angle between the penetrating needle 200 and the sheath 100. Therefore, the penetrating needle 200 is allowed to perform the ablation surgery on the affected part 40 in a more flexible manner.

Other embodiments are described below for illustrative purposes. It is to be noted that the following embodiments use the reference numerals and a part of the contents of the above embodiments, the same reference numerals are used to denote the same or similar elements, and the description of the same technical contents is omitted. For the description of the omitted part, reference may be made to the above embodiments, and details are not described in the following embodiments.

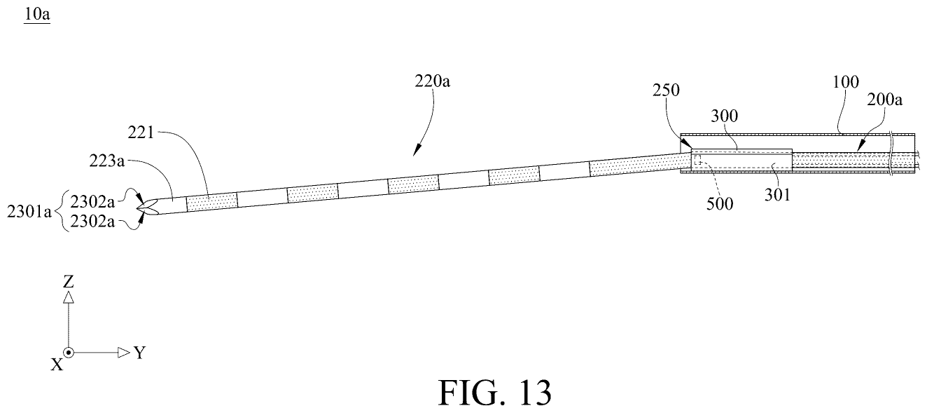

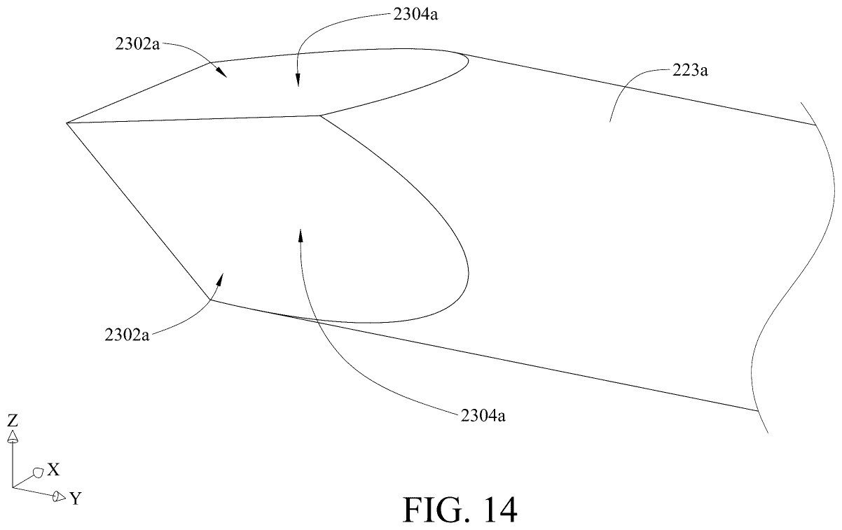

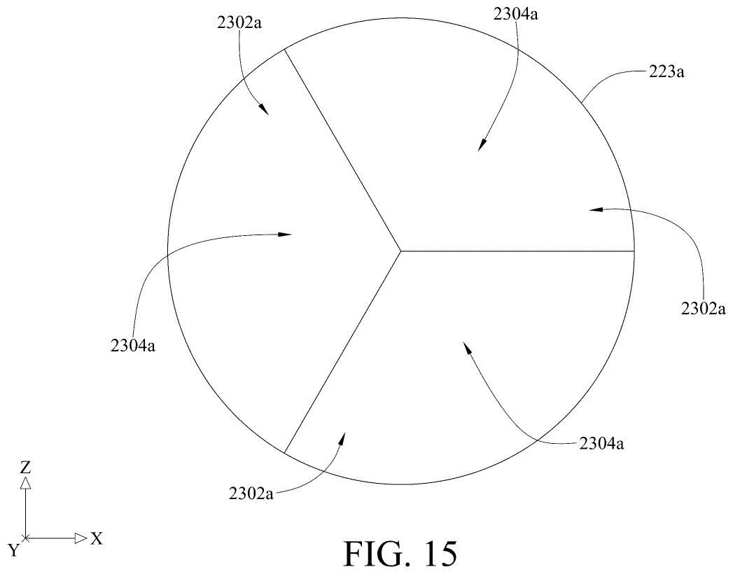

The disclosure is not limited by the structure of the penetrating portion. Please refer to FIGS. 13 to 15. FIG. 13 is a cross-sectional view of an ablation device 10a according to a second embodiment of the disclosure. FIG. 14 is a partially enlarged perspective view of the ablation device 10a in FIG. 13. FIG. 15 is a plan view of the ablation device 10a in FIG. 13 from another viewing angle. The difference between this embodiment and the first embodiment is in that: in a penetrating needle 200a of the ablation device 10a of this embodiment, a side of a penetrating portion 223a of a penetrating end part 220a located away from the flexible portion 221 has a positioning structure 2301a. The positioning structure 2301a is configured to be positioned at the affected part. In this embodiment, the positioning structure 2301a includes, for example, a plurality of first positioning recesses 2302a. A plurality of bottom surfaces 2304a of the first positioning recesses 2302a are connected to one another on an end of the penetrating portion 223a located away from the flexible portion 221. With the positioning of the first positioning recesses 2302a, the penetrating portion 223a is facilitated to be supported firmly to perform the ablation surgery by penetrating into the affected part.

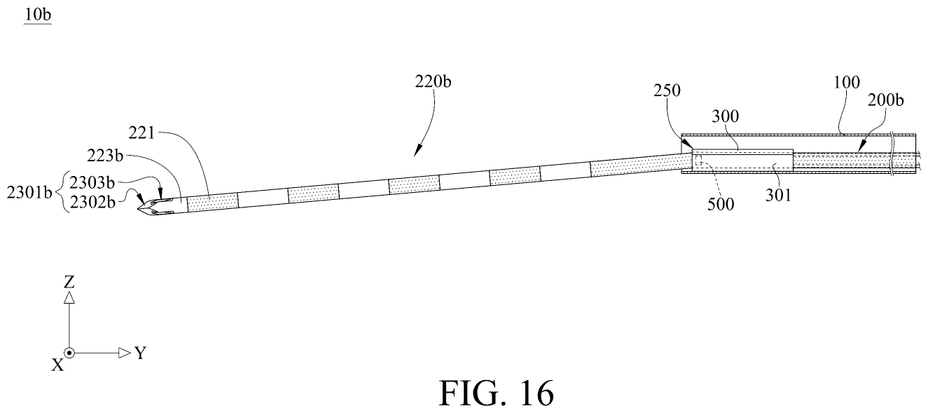

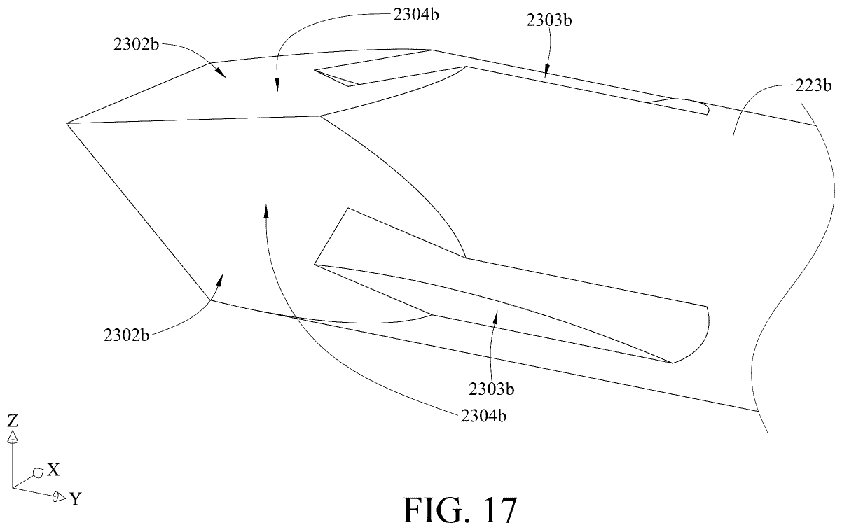

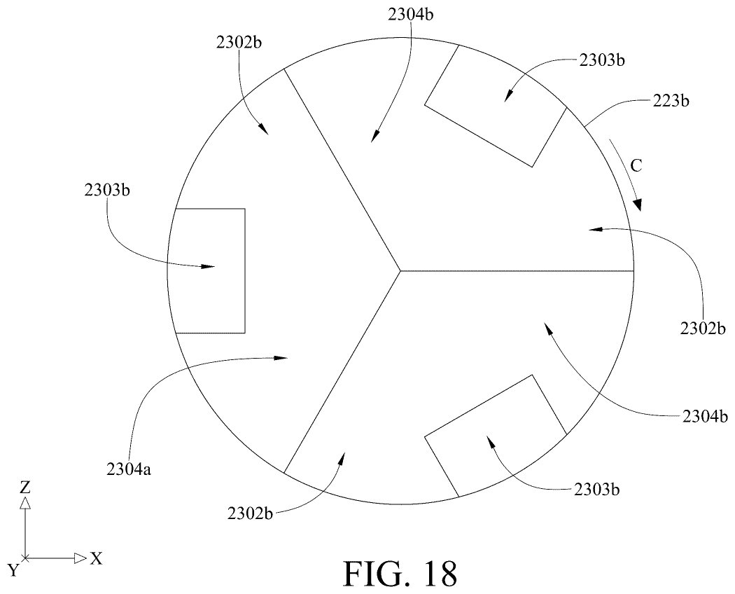

Alternatively, please refer to FIGS. 16 to 18. FIG. 16 is a cross-sectional view of an ablation device 10b according to a third embodiment of the disclosure. FIG. 17 is a partially enlarged perspective view of the ablation device 10b in FIG. 16. FIG. 18 is a plan view of the ablation device 10b in FIG. 16 from another viewing angle. The only difference between this embodiment and the second embodiment is the configuration of a positioning structure 2301b. In detail, in a penetrating needle 200b of the ablation device 10b, the positioning structure 2301b of a penetrating portion 223b of a penetrating end part 220b includes a plurality of first positioning recesses 2302b and a plurality of second positioning recesses 2303b. A plurality of bottom surfaces 2304b of the first positioning recesses 2302b are connected to one another on an end of the penetrating portion 223b located away from the flexible portion 221. The second positioning recesses 2303b are recessed radially and inwards from the penetrating portion 223b, and are arranged along the circumferential direction C of the penetrating portion 223b. With the positioning of the second positioning recesses 2303b, the penetrating portion 223b is facilitated to be supported firmly to perform the ablation surgery by penetrating into the affected part.

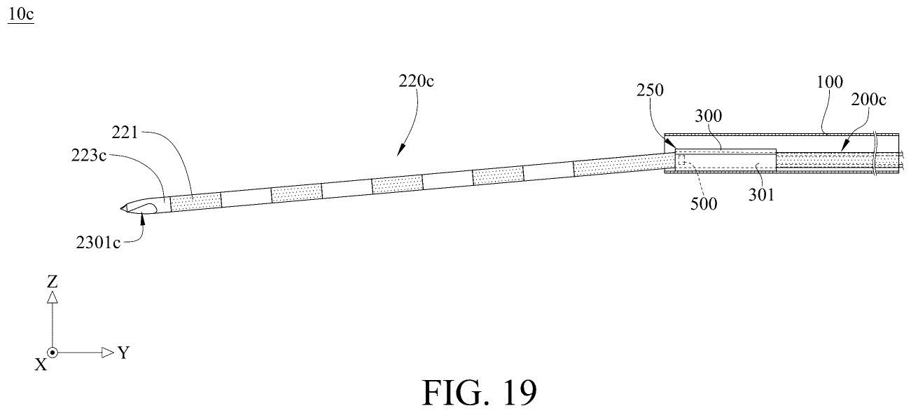

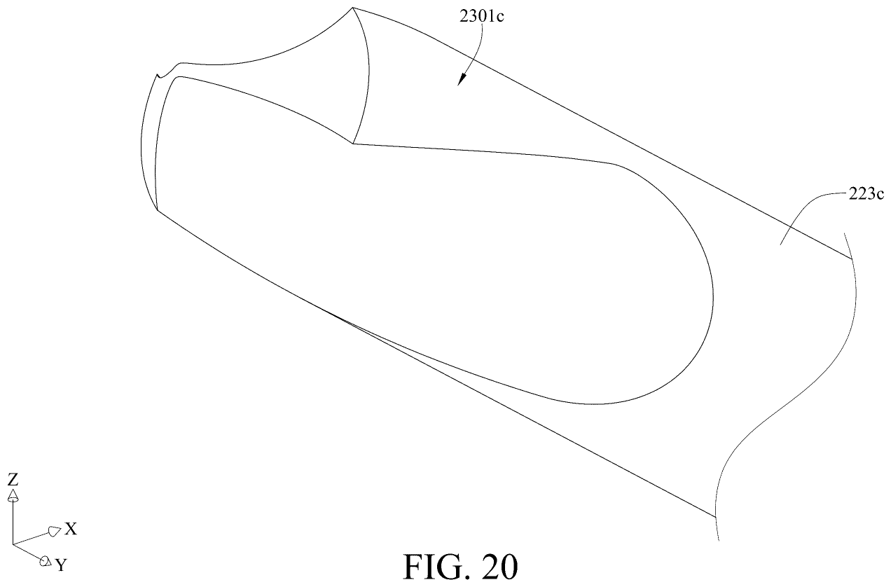

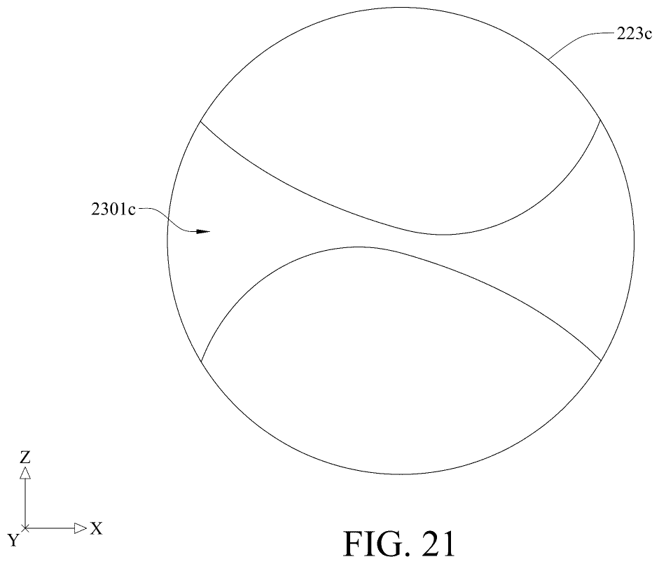

Alternatively, please refer to FIGS. 19 to 21. FIG. 19 is a cross-sectional view of an ablation device 10c according to a fourth embodiment of the disclosure. FIG. 20 is a partially enlarged perspective view of the ablation device 10c in FIG. 19. FIG. 21 is a plan view of the ablation device 10c in FIG. 19 from another viewing angle. The difference between this embodiment and the first embodiment is in that: in a penetrating needle 200c of the ablation device 10c of this embodiment, a side of a penetrating portion 223c of a penetrating end part 220c located away from the flexible portion 221 has a positioning structure 2301c. The positioning structure 2301c is, for example, a spiral structure and is configured to be positioned at the affected part. With the positioning of the positioning structure 2301c, the penetrating portion 223c is facilitated to be supported firmly to perform the ablation surgery by penetrating into the affected part.

According to the ablation device and the ablation system disclosed by above embodiments, a part of the penetrating needle is flexible. Thus, the ablation device is allowed to enter into the bronchus to perform the ablation surgery by cooperating with the endoscope. In this way, the ablation surgery is allowed to be performed without the assistance of the CT images, which prevents the patient from being exposed to the radiation. Moreover, the possibility of bringing the medical complication after performing the ablation by the ablation device is lowered. Also, the clamping component is disposed between the sheath and the penetrating needle to support the penetrating needle. Thus, even though a part of the penetrating needle is flexible, the clamping component still provide sufficient support, thereby allowing the penetrating needle to penetrate into the affected part to perform ablation surgery. In addition, the clamping component enables the adjustment of the penetrating angle of the penetrating needle, thereby allowing the penetrating needle to perform the ablation surgery on the affected part in a more flexible manner.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. An ablation device, configured to be cooperated with a sheath and penetrate into an affected part, the ablation device comprising:
a penetrating needle, movably disposed through the sheath, wherein a part of the penetrating needle is flexible; and
an angle adjusting assembly, comprising a clamping component disposed between the sheath and the penetrating needle;
wherein, the clamping component has a clamping state and a releasing state, when the clamping component is in the clamping state, the clamping component is configured to support the penetrating needle to allow the penetrating needle to be configured to penetrate into the affected part along an axial direction of the sheath, and the clamping component is configured to adjust a penetrating angle of the penetrating needle, when the clamping component is in the releasing state, the clamping component releases the penetrating needle.

2. The ablation device according to claim 1, wherein the clamping component has elasticity.

3. The ablation device according to claim 2, wherein the clamping component comprises at least one elastic bag, the at least one elastic bag is configured to be in fluid communication with a filling/discharging unit via at least one tube, the at least one elastic bag has a fixed surface and a movable surface facing away from each other, the fixed surface faces the sheath, the movable surface faces the penetrating needle, when the filling/discharging unit fills the at least one elastic bag via the at least one tube to allow the clamping component to be in the clamping state, the at least one elastic bag expands and fills a gap between the sheath and the penetrating needle, and the fixed surface and the movable surface rest on the sheath and the penetrating needle, respectively, so as to allow the clamping component to support the penetrating needle, when the filling/discharging unit releases the at least one elastic bag via the at least one tube to allow the clamping component to be in the releasing state, the at least one elastic bag is released and a part of the fixed surface is spaced apart from the sheath, so as to allow the clamping component to release the penetrating needle.

4. The ablation device according to claim 3, wherein the at least one elastic bag is sleeved on the penetrating needle.

5. The ablation device according to claim 4, wherein the at least one elastic bag comprises a plurality of elastic bags, the plurality of elastic bags are arranged along a circumferential direction of the penetrating needle, and a plurality of chambers of the plurality of elastic bags are not in fluid communication with each other.

6. The ablation device according to claim 5, wherein the plurality of elastic bags together surround the penetrating needle.

7. The ablation device according to claim 1, further comprising a pressure sensor disposed on a side of the clamping component located close to the sheath.

8. The ablation device according to claim 1, further comprising a temperature sensor disposed on a side of the clamping component.

9. The ablation device according to claim 1, wherein the penetrating needle has an assembling end part and a penetrating end part connected to each other, the assembling end part is movably disposed in the sheath, the penetrating end part comprises at least one flexible portion, at least one rigid portion and a penetrating portion, the at least one flexible portion and the at least one rigid portion are alternately arranged and connected along an axial direction of the penetrating end part, and the penetrating portion protrudes from the at least one flexible portion and is configured to penetrate into the affected part.

10. The ablation device according to claim 9, wherein the at least one flexible portion is electrically insulating, and the at least one rigid portion is electrically conductive.

11. The ablation device according to claim 9, wherein a side of the penetrating portion located away from the at least one flexible portion has a positioning structure configured to be positioned at the affected part.

12. The ablation device according to claim 11, wherein the positioning structure comprises a plurality of first positioning recesses, a plurality of bottom surfaces of the plurality of first positioning recesses are connected to each other on an end of the penetrating portion located away from the at least one flexible portion.

13. The ablation device according to claim 12, wherein the positioning structure further comprises a plurality of second positioning recesses, the plurality of second positioning recesses are recessed radially and inwards from the penetrating portion, and are arranged along a circumferential direction of the penetrating portion.

14. The ablation device according to claim 11, wherein the positioning structure is a spiral structure.

15. An ablation system, configured to be cooperated with a sheath and penetrate into an affected part, the ablation system comprising;
a control module;
an ablation apparatus, electrically connected to the control module; and
an ablation device, comprising:
a penetrating needle, movably disposed through the sheath, wherein a part of the penetrating needle is flexible, the penetrating needle is electrically connected to the ablation apparatus, and the control module is configured to control the ablation apparatus to send an ablation signal to the penetrating needle; and
an angle adjusting assembly, comprising a clamping component disposed between the sheath and the penetrating needle;
wherein, the clamping component has a clamping state and a releasing state, when the clamping component is in the clamping state, the clamping component is configured to support the penetrating needle to allow the penetrating needle to be configured to penetrate into the affected part along an axial direction of the sheath, and the clamping component is configured to adjust a penetrating angle of the penetrating needle, when the clamping component is in the releasing state, the clamping component releases the penetrating needle.

16. The ablation system according to claim 15, further comprising a robot arm electrically connected to the control module and configured to move the penetrating needle or the sheath.

17. The ablation system according to claim 15, wherein the clamping component has elasticity.

18. The ablation system according to claim 17, further comprising a filling/discharging unit, wherein the filling/discharging unit is electrically connected to the control module, the clamping component comprises at least one elastic bag, the at least one elastic bag is configured to be in fluid communication with a filling/discharging unit via at least one tube, the at least one elastic bag has a fixed surface and a movable surface facing away from each other, the fixed surface faces the sheath, the movable surface faces the penetrating needle, when the filling/discharging unit fills the at least one elastic bag via the at least one tube to allow the clamping component to be in the clamping state, the at least one elastic bag expands and is filled in a gap between the sheath and the penetrating needle, and the fixed surface and the movable surface rest on the sheath and the penetrating needle, respectively, so as to allow the clamping component to support the penetrating needle, when the filling/discharging unit releases the at least one elastic bag via the at least one tube to allow the clamping component to be in the releasing state, the at least one elastic bag is released and a part of the fixed surface is spaced apart from the sheath, so as to allow the clamping component to release the penetrating needle.

19. The ablation system according to claim 18, wherein the at least one elastic bag is sleeved on the penetrating needle.

20. The ablation system according to claim 19, wherein the at least one elastic bag comprises a plurality of elastic bags, the plurality of elastic bags are arranged along a circumferential direction of the penetrating needle, and a plurality of chambers of the plurality of elastic bags are not in fluid communication with each other.
